(19) United States
(12) Patent Application Publication
(10) Pub. No.: US 2010/0305529 A1
(43) Pub. Date: Dec. 2, 2010

(54) Absorbent Article With Absorbent Polymer Material, Wetness Indicator, And Reduced Migration Of Surfactant

(76) Inventors: Gregory Ashton, Cincinnati, OH (US); Masaharu Nishikawa, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/476,272

(22) Filed: Jun. 2, 2009

(51) Int. Cl. A61F 13/42; A61F 13/53; A61F 13/15

(52) U.S. Cl. 604/361; 604/367; 604/370; 604/374

(57) ABSTRACT

This disclosure provides for an absorbent article having an absorbent polymer material and a component comprising a hydrophilic surfactant-treated nonwoven material, and/or a wetness indicator. Various stabilizing components and methods are disclosed for stabilizing the surfactant-treated synthetic fibers in the nonwoven material so as to reduce surfactant loss during wetting, and enhance the performance of the absorbent article.

### FIELD OF THE INVENTION

The present disclosure generally relates to an absorbent article, and more particularly to a disposable absorbent article with absorbent polymer material, such as a diaper.

### BACKGROUND OF THE INVENTION

Absorbent articles, such as disposable diapers, training pants, and adult incontinence undergarments, absorb and contain body exudates. They also are intended to prevent body exudates from soiling or wetting clothing or other articles such as bedding that may come in contact with the wearer. A disposable absorbent article such as a disposable diaper may be worn for several hours in a dry state or in a urine loaded state. Therefore, efforts have been made toward improving the fit and comfort of the absorbent article to the wearer, both when the article is dry and when fully or partially loaded with liquid exudate, while maintaining the absorption and containment functions of the article.

Some absorbent articles, like diapers, contain an absorbent polymer material, such as an absorbent particulate polymer material, as a component of an absorbent core, which may swell or gel upon wetting as it absorbs liquid. Nonwoven fibrous structures are also in common use in absorbent articles, for example, in topsheet material or as a core cover to enclose the absorbent core and provide structural integrity when the absorbent core is wet or dry. Certain natural fibers and a variety of synthetic fibers have been employed in these applications, with the synthetic fibers seeing increased utility. Synthetic fibers may have certain advantages over natural fibers, such as their low flexural rigidity which may increase product softness; however, the use of synthetic fibers can have certain limitations. For example, synthetic fibers typically have the general characteristic of being hydrophobic, while in many absorbent article applications such as hygiene products, it is desirable that the fibrous structures be hydrophilic. Therefore, nonwoven fibrous structures such as a core cover may need to be rendered hydrophilic to attain improvements in their desired function.

One method of imparting hydrophilic properties to nonwoven fibrous structures is to contact or coat the surface of the nonwoven structures with a hydrophilic surfactant. As this coating does not always lead to a strong chemical bond between the nonwoven material and the surfactant, the surfactant may be eluted or washed off when the absorbent article is wetted during use. When this occurs, the absorbent properties of the nonwoven structure may be adversely affected, resulting in a performance loss during use on diapers or other articles comprising such fibrous structures. Moreover, if the eluted surfactant migrates or diffuses through the absorbent article to materials that were selected for their hydrophobic barrier properties, these hydrophobic materials may become hydrophilic, further reducing the performance of the article by loss of their barrier properties.

In a further aspect, because a disposable absorbent article such as a disposable diaper may be worn for several hours when the article is fully or partially loaded with liquid exudate, a mechanism to communicate to the caregiver if the absorbent article is still useful for providing absorbency would be helpful. For example, a visual signal that could indicate both when the article is dry and when the article is loaded with liquid exudate would be a useful feature.

### SUMMARY OF THE INVENTION

The present disclosure provides a disposable absorbent article which can comprise a chassis and an absorbent core, which may address one or more technical problems described above. In one aspect, the chassis may contain a liquid pervious topsheet and a liquid impervious backsheet that is at least partially joined to the topsheet. Further, the absorbent core can be disposed at least partially between the topsheet and the backsheet and can comprise an absorbent polymer material. In one aspect, the disposable absorbent article or any component thereof also comprises a hydrophilic nonwoven material comprising a plurality of surfactant-treated synthetic fibers. In another aspect, the disposable absorbent article can further comprise a stabilizing component which reduces the loss of surfactant from the hydrophilic nonwoven material during wetting. While a variety of stabilizing component can be employed, in one aspect, the stabilizing component can be selected from: i) at least one substantially permanent hydrophilizing agent associated with the hydrophilic nonwoven material; or at least one substantially permanent hydrophobic agent independent of the hydrophilic nonwoven material; or a combination of i) and ii). Moreover, the disposable absorbent article also may include a wetness indicator disposed between the absorbent core and the backsheet and in liquid communication with the absorbent core, in which the wetness indicator includes at least one responsive color composition such that, upon wetting, a visible change occurs.

According to this disclosure, the disposable absorbent article can further include at least one of: a) a core cover contiguous with the absorbent core and disposed between the topsheet and the absorbent core; b) a dusting cover contiguous with the absorbent core and disposed between the backsheet and the absorbent core; c) an acquisition system disposed between the topsheet and the absorbent core, or d) any combination thereof.

According to another aspect of this disclosure, a method of a stabilizing surfactant-treated synthetic fibers to surfactant loss during wetting is provided, the method comprising: a) providing a disposable absorbent article comprising a hydrophilic nonwoven material, the nonwoven material comprising surfactant-treated synthetic fibers; b) combining or otherwise associating at least one substantially permanent hydrophilizing agent with the hydrophilic nonwoven material; and/or c) depositing at least one substantially permanent hydrophobic agent on a portion of the disposable absorbent article independent of the hydrophilic nonwoven material.

These and other features and advantages provided by this disclosure may be apparent from reading the following detailed description, drawings, and claims.

### BRIEF DESCRIPTION OF THE DRAWINGS

FIG. 1 illustrates one possible aspect of the association of a dimeric hydrophilizing agent and a synthetic fiber.

FIG. 2 is a plan view of a diaper in accordance with one aspect of the present disclosure.

FIG. 3 is a cross sectional view of the diaper shown in FIG. 2 taken along the sectional line 2-2 of FIG. 2.

FIGS. 4A and 4B illustrate the absorbent article of the present disclosure having a wetness indicator that changes color upon wetting, with FIG. 4A illustrating prior to wetting and 4B illustrating after wetting.

FIGS. 5A and 5B illustrate an absorbent article of the present disclosure having a wetness indicator wherein the background graphic is a series of large and small bubbles that upon wetting becomes a series of raindrops and small bubbles.

FIGS. 6A and 6B illustrate an absorbent article of the present disclosure having a wetness indicator wherein the background graphic of small and large bubbles in series changes to raindrops within the large bubbles, upon wetting.

### DETAILED DESCRIPTION OF THE INVENTION

“Absorbent article” refers to devices that absorb and contain body exudates, and, more specifically, refers to devices that are placed against or in proximity to the body of the wearer to absorb and contain the various exudates discharged from the body. Absorbent articles may include diapers, training pants, adult incontinence undergarments, feminine hygiene products, breast pads, care mats, bibs, wound dressing products, and the like. As used herein, the term “body fluids” or “body exudates” includes, but is not limited to, urine, blood, vaginal discharges, breast milk, sweat and fecal matter.

“Absorbent core” means a structure typically disposed between a topsheet and backsheet of an absorbent article for absorbing and containing liquid received by the absorbent article and may comprise one or more substrates, absorbent polymer material disposed on the one or more substrates, and a thermoplastic composition on the absorbent polymer material and at least a portion of the one or more substrates for immobilizing the absorbent polymer material on the one or more substrates. In a multilayer absorbent core, the absorbent core may also include a cover layer. The one or more substrates and the cover layer may comprise a nonwoven. Further, the absorbent core is substantially cellulose free. The absorbent core does not include an acquisition system, a topsheet, or a backsheet of the absorbent article. In a certain embodiment, the absorbent core would consist essentially of the one or more substrates, the absorbent polymer material, the thermoplastic composition, and optionally the cover layer.

“Absorbent polymer material,” “absorbent gelling material,” “AGM,” “superabsorbent,” and “superabsorbent material” are used herein interchangeably and refer to cross linked polymeric materials that can absorb at least 5 times their weight of an aqueous 0.9% saline solution as measured using the Centrifuge Retention Capacity test (Edana 441.2-01); “Absorbent particulate polymer material” is used herein to refer to an absorbent polymer material which is in particulate form so as to be flowable in the dry state.

“Comprise,” “comprising,” and “comprises” are open ended terms, each specifies the presence of what follows, e.g., a component, but does not preclude the presence of other features, e.g., elements, steps, components known in the art, or disclosed herein.

“Consisting essentially of” is used herein to limit the scope of subject matter, such as that in a claim, to the specified materials or steps and those that do not materially affect the basic and novel characteristics of the subject matter.

“Disposable” is used in its ordinary sense to mean an article that is disposed or discarded after a limited number of usage events over varying lengths of time, for example, less than about 20 events, less than about 10 events, less than about 5 events, or less than about 2 events.

“Diaper” refers to an absorbent article generally worn by infants and incontinent persons about the lower torso so as to encircle the waist and legs of the wearer and that is specifically adapted to receive and contain urinary and fecal waste. As used herein, term “diaper” also includes “pants” which is defined below.

“Fiber” and “filament” are used interchangeably.

A “hydrophilizing agent” may be broadly disclosed as comprising oligomeric or polymeric “backbones” to which are appended hydrophilic substituents. In this aspect, “oligomeric” herein refers to a polymer molecule with fewer than 10 repeating units such as dimers, trimers, tetramers, etc., whereas “polymeric” herein refers to a molecule with greater than 10 repeating units. A variety of such agents have found utility as soil release compounds in the detergency arts. The present methods and articles disclosed herein can employ such compounds in the stabilized nonwoven fibrous structure described herein. Such compounds are usually water-soluble or water-dispersible under the typical usage conditions disclosed herein, e.g., in a fiber slurry comprising an aqueous carrier medium; from about 20° C. to about 90° C. operating conditions; usage levels of about 0.001% to about 20%, by weight of the fiber weight; weight ratio of hydrophilizing agent to hydrophobic fiber in slurry in the range of from about 0.0001:1 to about 1:1. Further aspects of the hydrophilizing agents may be found in WO 07/123702, published Nov. 1, 2007.

A “nonwoven” is a manufactured sheet, web or batt of directionally or randomly orientated fibers, bonded by friction, and/or cohesion and/or adhesion, excluding paper and products which are woven, knitted, tufted, stitch-bonded incorporating binding yarns or filaments, or felted by wet-milling, whether or not additionally needled. The fibers may be of natural or man-made origin and may be staple or continuous filaments or be formed in situ. Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yam). Nonwoven fabrics can be formed by many processes such as meltblowing, spunbonding, solvent spinning, electrospinning, and carding. The basis weight of nonwoven fabrics is usually expressed in grams per square meter (gsm).

“Pant” or “training pant”, as used herein, refer to disposable garments having a waist opening and leg openings designed for infant or adult wearers. A pant may be placed in position on the wearer by inserting the wearer's legs into the leg openings and sliding the pant into position about a wearer's lower torso. A pant may be preformed by any suitable technique including, but not limited to, joining together portions of the article using refastenable and/or non-refastenable bonds (e.g., seam, weld, adhesive, cohesive bond, fastener, etc.). A pant may be preformed anywhere along the circumference of the article (e.g., side fastened, front waist fastened). While the terms “pant” or “pants” are used herein, pants are also commonly referred to as “closed diapers,” “prefastened diapers,” “pull-on diapers,” “training pants,” and “diaper-pants”. Suitable pants are disclosed in U.S. Pat. No. 5,246,433, issued to Hasse, et al. on Sep. 21, 1993; U.S. Pat. No. 5,569,234, issued to Buell et al. on Oct. 29, 1996; U.S. Pat. No. 6,120,487, issued to Ashton on Sep. 19, 2000; U.S. Pat. No. 6,120,489, issued to Johnson et al. on Sep. 19, 2000; U.S. Pat. No. 4,940,464, issued to Van Gompel et al. on Jul. 10, 1990; U.S. Pat. No. 5,092,861, issued to Nomura et al. on Mar. 3, 1992; U.S. Patent Publication No. 2003/0233082 A1, entitled “Highly Flexible And Low Deformation Fastening Device”, filed on Jun. 13, 2002; U.S. Pat. No. 5,897,545, issued to Kline et al. on Apr. 27, 1999; U.S. Pat. No. 5,957,908, issued to Kline et al on Sep. 28, 1999.

“Substantially permanent” means that the treatment, additive, or condition at issue retains its efficacy for most of the useful life of the disposable absorbable article. For example, this term can be used to describe a treatment, additive, or condition that provides the desired effect for at least about 60% of the useful life of the disposable absorbent article. “Substantially permanent” can be used to describe a treatment, additive, or condition that provides the desired effect for at least about 70%, at least about 80%, at least about 90%, or at least about 95% of the useful life of the disposable absorbent article.

“Thickness” and “caliper” are used herein interchangeably.

In one aspect, this disclosure provides for a method of a stabilizing surfactant-treated synthetic fibers so as to reduce surfactant loss during wetting, in which the synthetic fibers are provided in a hydrophilic nonwoven material in a disposable absorbent article. In one aspect, the fibers or the hydrophilic nonwoven material containing the fibers may be treated with, combined with, or otherwise associating at least one substantially permanent hydrophilizing agent, such that the hydrophilic character thereof is sustained during and after wetting. In another aspect of this method, at least one substantially permanent hydrophobic agent may be added to, coated on, or deposited on a portion of the disposable absorbent article, independent of the hydrophilic nonwoven material that contains the surfactant-treated synthetic fibers, so that migration of any surfactant to that portion of the disposable absorbent article independent of the hydrophilic nonwoven material during wetting is reduced. Thus, while not bound by theory, it is thought that if eluted surfactant migrates or diffuses through the absorbent article to materials that selected for their hydrophobic barrier properties, these hydrophobic materials may become hydrophilic, further reducing the performance of the article by loss of their barrier properties. Thus, these aspects of more permanently hydrophilizing the surfactant-treated synthetic fibers and hydrophilic nonwoven material containing these fibers, and more permanently enhancing the hydrophobic properties of the portions of the disposable absorbent article independent of the hydrophilic nonwoven material constitute approaches in this surfactant migration attenuation system disclosed herein.

According to one aspect, this disclosure provides a disposable absorbent article, comprising: a) a chassis comprising a liquid pervious topsheet and a liquid impervious backsheet that is at least partially joined to the topsheet; b) an absorbent core disposed at least partially between the topsheet and the backsheet and comprising an absorbent polymer material; wherein the disposable absorbent article or any component thereof comprises a hydrophilic nonwoven material comprising a plurality of surfactant-treated synthetic fibers; the disposable absorbent article further comprising: c) a stabilizing component which reduces the loss of surfactant from the hydrophilic nonwoven material during wetting, the stabilizing component selected from: i) at least one substantially permanent hydrophilizing agent associated with the hydrophilic nonwoven material; ii) at least one substantially permanent hydrophobic agent independent of the hydrophilic nonwoven material, or iii) a combination of i) and ii); and d) a wetness indicator disposed between the absorbent core and the backsheet and in liquid communication with the absorbent core, the wetness indicator comprises at least one responsive color composition such that, upon wetting, a visible change occurs.

In a further aspect of this disclosure, the stabilizing component generally can comprise or can be selected from: a) a hydrophilizing agent durably associated with the nonwoven; b) a hydrophobic stabilizing component; a nanofiber nonwoven stabilizing component; or d) any combination thereof. Each of these stabilizing components is described hereinbelow. The use of these stabilizing components and wetness indicators is illustrated using a diaper as an example of a disposable absorbent article.

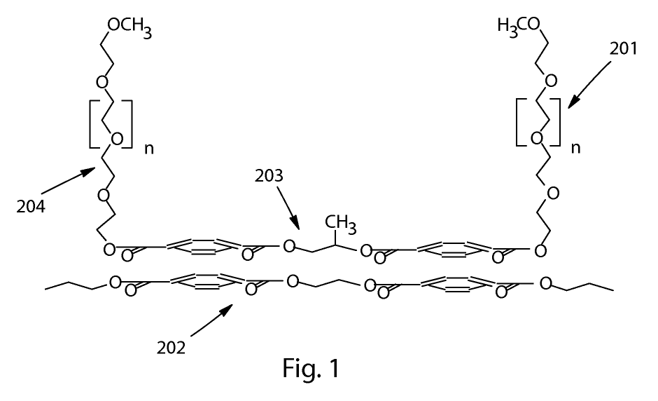

In one aspect of stabilizing the nonwoven material to surfactant loss, the stabilizing component can be a substantially permanent hydrophilizing agent. For example, the synthetic fibers of the nonwoven material may contain a polymer and a hydrophilizing agent, wherein the polymer and the hydrophilizing agent comprise complementary segments that are associated with one another. FIG. 1 is illustrative of this aspect, but is not intended to be limiting. FIG. 1 illustrates an artist's conception at the molecular level of a hydrophilizing agent 201 having a dimeric “backbone,” a complementary segment 203, and hydrophilic substituents 204 associated with a complementary segment of a synthetic fiber 202, wherein n may be from about 1 to about 15.

While not intending to be limited by theory, it is surmised that the hydrophilizing agent can become associated with one or more surfaces of the hydrophobic synthetic fiber. The association between the hydrophilizing agent and the synthetic fiber may be a durable association, and it may provide for the synthetic fibers to exhibit hydrophilic characteristics as opposed to the hydrophobic characteristics displayed by the synthetic fibers alone. It is further surmised in theory that the hydrophobicity of synthetic fibers alone may generally cause the synthetic fibers to clump together during the webmaking process or within a fibrous structure. Regardless of the reason, it has now been found that the association of a hydrophilizing agent with the synthetic fibers may provide for the dispersion of the synthetic fibers in a fibrous structure. For example, during a wet laid papermaking process, there may be a dispersion of the synthetic fibers in a fluid carrier which may then promote the dispersion of the synthetic fibers in the fibrous structure. Natural fibers may optionally be present in the dispersion as the natural fibers may not interfere with the association of the hydrophilizing agent to the synthetic fibers. The hydrophilizing agent may associate with the natural fibers; however, this association will not prevent the hydrophilizing agent from associating with the synthetic fibers.

Hydrophilizing agents can include a variety of charged anionic or cationic species as well as noncharged monomer units. The anionic and cationic polymers may enhance both the deposition and the wettability of the synthetic fibers. Examples of hydrophilizing agents comprising cationic functionalities are disclosed in U.S. Pat. No. 4,956,447. The structure of the hydrophilizing agents maybe linear, branched or even star-shaped. Structures and charge distributions may be tailored for application to different fiber or textile types.

The hydrophilizing agent may associate with the synthetic fibers by a correspondence between the hydrophilizing agent and the surface characteristics of the synthetic fibers. This correspondence may be based on physical characteristics of the synthetic fibers and hydrophilizing agent. Such physical characteristics may include, but are not limited to, degree of crystallinity and molecular weight. Correspondence between the physical characteristics of the hydrophilizing agents and the synthetic fibers may aid in the durability of the association formed between the hydrophilizing agents and the synthetic fibers. It has been found that an association based upon physical characteristics may be durable wherein the hydrophilizing agent may not “wash off from the synthetic fibers. As such, the hydrophilizing agents of the present disclosure may be distinguished from typical surfactants. The bond between the synthetic fibers and the hydrophilizing agent may be durable, and the synthetic fibers may exhibit a durable wettability.

In one aspect, the synthetic fibers may exhibit a mean contact angle of less than about 72°. The synthetic fibers may exhibit a mean contact angle of less than about 72° and after a 10 minute water wash the mean contact angle of the synthetic fibers may remain below about 72°. The synthetic fibers may exhibit a mean contact angle following a 10 minute water wash of less than about 66°, 63°, 60°, 55° or 50°. The synthetic fibers exhibiting such mean contact angles may be associated with a hydrophilizing agent. The bond between the synthetic fibers and the hydrophilizing agent may be durable and the hydrophilizing agent may not be washed off the synthetic fibers after a single insult of fluid. A surfactant, on the other hand, typically is unable to form such a durable bond and may be washed off the synthetic fibers upon a single insult of fluid. Furthermore, a fibrous structure comprising synthetic fibers and a hydrophilizing agent may demonstrate a sustainable wettability, as detailed herein, whereas a fibrous structure comprising synthetic fibers and a surfactant may not exhibit a sustainable wettability. A more permanent association may be made between the hydrophilizing agent and the synthetic fibers by heating the combination of the hydrophilizing agent and the synthetic fibers above the melting temperature of the hydrophilizing agent. However, a surfactant may be used along with or in addition to the hydrophilizing agents disclosed herein.

Hydrophilizing agents may comprise greater than about 3 ppm of a hydrophilizing agent/synthetic fiber and/or natural fiber combination. Hydrophilizing agents may generally comprise from about 10 ppm, about 20 ppm, about 30 ppm, or about 40 ppm to about 50 ppm, about 60 ppm, about 80 ppm, or about 100 ppm of a hydrophilizing agent/synthetic fiber and/or natural fibers combination. In one aspect, the compositions disclosed herein may comprise greater than about 0.001% of a hydrophilizing agent. The compositions herein may comprise from about 0.001% to about 2%, 5%, 10% or 20% of a hydrophilizing agent.

In another aspect, the hydrophilizing agent may comprise a segment that may be complementary to the polymer of the synthetic fibers. The complementary segment may comprise a polyester segment, and in one aspect, the polyester segment may comprise a polyethylene terephthalate segment. Further, the hydrophilizing agent may be oligomeric or polymeric. For example, the hydrophilizing agent may be a copolymer of ethoxylate siloxane. The hydrophilizing agent also may be soil release agent. Such a hydrophilizing agent may be a polymer. Polymeric hydrophilizing agents useful in the present disclosure may include, but are not limited to, polyester, poly(ethoxylate), polyethylene oxide, polyoxyethylene, polyethylene glycol, polypropylene glycol, terephthalate, polypropylene oxide, polyethylene terephthalate, polyoxyethylene terephthalate, ethoxylate siloxane, or any combination thereof. Polyesters of terephthalic and other aromatic dicarboxylic acids having soil release properties such as polyethylene terephthalate/polyoxyethylene terephthalate and polyethylene terephthalate/polyethylene glycol polymers, among other polyester polymers, may be utilized as the hydrophilizing agent in the fibrous structure. As noted above, a wide variety of hydrophilizing agents, also known as SRP's, SRA's, and SRE's, are well-recognized materials in the detergency arts.

Higher molecular weight (e.g., 40,000 to 50,000 M.W.) polyesters containing random or block ethylene terephthalate/polyethylene glycol (PEG) terephthalate units have been used as soil release agents in laundry cleaning compositions. For example, see U.S. Pat. Nos. 3,893,929; 3,959,230; and 3,962,152. Sulfonated linear terephthalate ester oligomers are disclosed in U.S. Pat. No. 4,968,451. Nonionic end-capped 1,2-propylene/polyoxyethylene terephthalate polyesters are disclosed in U.S. Pat. No. 4,711,730 and nonionic-capped block polyester oligomeric compounds are disclosed in U.S. Pat. No. 4,702,857. Partly- and fully- anionic-end-capped oligomeric esters are disclosed further in U.S. Pat. No. 4,721,580 and anionic, especially sulfoaroyl, end-capped terephthalate esters are disclosed in U.S. Pat. No. 4,877,896 and U.S. Pat. No. 5,415,807.

U.S. Pat. No. 4,427,557 discloses low molecular weight copolyesters (M.W. 2,000 to 10,000) which can be used in aqueous dispersions to impart soil release properties to polyester fibers. The copolyesters are formed by the reaction of ethylene glycol, a PEG having an average molecular weight of 200 to 1000, an aromatic dicarboxylic acid (e.g., dimethyl terephthalate), and a sulfonated aromatic dicarboxylic acid (e.g., dimethyl 5-sulfoisophthalate). The PEG can be replaced in part with monoalkylethers of PEG such as the methyl, ethyl and butyl ethers.

A hydrophilizing agent may be a copolymer having blocks of terephthalate and polyethylene oxide. More specifically, these polymers may comprise repeating units of ethylene and/or propylene terephthalate and polyethylene oxide terephthalate at a molar ratio of ethylene terephthalate units to polyethylene oxide terephthalate units of from about 25:75 to about 35:65, wherein the polyethylene oxide terephthalate contains polyethylene oxide blocks having molecular weights of from about 300 to about 2000. The molecular weight of this polymeric hydrophilizing agent may be in the range of from about 5,000 to about 55,000.

Another polymeric hydrophilizing agent may be a crystallizable polyester with repeat units of ethylene terephthalate units comprising from about 10% to about 15% by weight of ethylene terephthalate units together with from about 10% to about 50% by weight of polyoxyethylene terephthalate units, derived from a polyoxyethylene glycol of average molecular weight of from about 300 to about 6,000, and the molar ratio of ethylene terephthalate units to polyoxyethylene terephthalate units in the crystallizable polymeric compound may be between 2:1 and 6:1. Examples of this polymer include, but are not limited to, the commercially available materials ZELCON® 4780 (from DuPont) and MILEASE® T (from ICI).

In another aspect, the poly(ethoxylate) regions may be tailored to have from about 1 to about 9, 1 to about 12, or 1 to about 15 ethoxylated groups and any other number of ethoxylated groups within the range of from about 1 to about 15. The number of poly(ethoxylated) regions maybe tailored to enhance the wettability of the synthetic fibers. Wettability of the synthetic fibers may be increased as the number of ethoxylated groups increases in the poly(ethoxylate) regions. Optionally, additional copolymers such as, but not limited to, polyethylene glycol and polypropylene glycol, may be used to control the crystallinity of the hydrophilizing agents.

In an alternative aspect, the hydrophilizing agents provided by the disclosure may be illustrated by one comprising from about 25% to about 100% by weight of an ester having the empirical formula (CAP)ₓ(EG/PG)_{y′}(OEG)_{y″}(PEG)_{y′″}(T)_{z}(SIP)q; wherein (CAP) represents the sodium salt form of the end-capping units i); (EG/PG) represents the oxyethyleneoxy and oxy-1,2-propyleneoxy units ii); (DEG) represents the di(oxyethylene)oxy units iii); (PEG) represents the poly(oxyethylene)oxy units iv); (T) represents the terephthaloyl units v); (SIP) represents the sodium salt form of 5-sulfoisophthaloyl units vi); x is from about 1 to 2; y′ is from about 0.5 to about 66; y″ is from 0 to about 50; y′″ is from 0 to about 50; y′+y″+y′″ totals are from about 0.5 to about 66; z is from about 1.5 to about 40; and q is from about 0.05 to about 26; wherein x, y′, y″, y′″, z and q represent the average number of moles of the corresponding units per mole of the ester. Hydrophilizing agents may be those wherein at least about 50% by weight of the ester has a molecular weight ranging from about 500 to about 5,000.

In one aspect, the hydrophilizing agents may have oxyethyleneoxy:oxy-1,2-propyleneoxy mole ratio ranges from about 0.5:1 to about 10:1; x is about 2, y′ is from about 2 to about 27, z is from about 2 to about 20, and q is about 0.4 to about 8. In yet another aspect, x is about 2, y′ is about 5, z is about 5, and q is about 1.

The hydrophilizing agents may associate with the synthetic fiber surface during the process of re-pulping the fibers. The synthetic fibers may also be provided with a finishing coat of the hydrophilizing agent prior to re-pulping the fibers. Additionally, the hydrophilizing agent may associate with the synthetic fibers as a melt-additive prior to extrusion of the synthetic fibers.

Additional disclosure relating to hydrophilizing agents may be found in U.S. Pat. Nos. 4,702,857; 4,861,512; 5,574,179 and 5,843,878. General methods of making a fibrous structure using hydrophilizing agents may be found in WO 07/123702, published Nov. 1, 2007.

In a further aspect of stabilizing the nonwoven material toward surfactant loss, the stabilizing component can be selected from at least one substantially permanent hydrophobic agent independent of or apart from the surfactant-treated hydrophilic nonwoven material. In this aspect, the substantially permanent hydrophobic agent typically is not associated with the fibers of the nonwoven material in the same manner disclosed for the hydrophilizing agent. However, the barrier material may constitute at least a partial coating or barrier material on the hydrophilic nonwoven material itself to reduce surfactant loss. The barrier material also may be disposed as at least a partial coating or barrier material on a hydrophobic material for which maintaining hydrophobic properties are desired, such as a barrier cuff. In this aspect, such barrier material may reduce the risk that these hydrophobic materials may become hydrophilic as a result of surfactant migration, which may further reduce the performance of the absorbent article. Moreover, the barrier material may be situated as a partial coating or barrier material between the surfactant-treated hydrophilic nonwoven material that may be a source of surfactant during wetting, and the hydrophobic site such as a barrier cuff.

A wide range of barrier materials or combinations of barrier materials can be employed in this aspect of stabilizing the surfactant-treated nonwoven material to surfactant loss. Moreover, these barrier materials can be applied on the nonwoven materials themselves or on the finished product using a spray process, kiss roll process, or the like. Treatments for stabilizing the nonwoven material to surfactant loss may include chemical, radiation, plasma or combinations thereof Further, the surface treatment to modify the surface characteristics may be accomplished by a coating on the surface, by pre-blending with a suitable hydrophobic barrier material or by incorporating a hydrophobic barrier material in-situ, which blooms to the surface by further processing.

In one aspect, fluorocarbon treatments of the nonwoven or other material provides the desired hydrophobicity such that the nonwoven or other material exhibits the desired water resistance characteristics to prevent loss or accumulation of surfactant, measured, for example, by the hydrohead test. In another aspect, fluorocarbon treatment using plasma or like technology can provide a very thin, hydrophobic coating such that the air permeability of the treated nonwoven is substantially unchanged. If desired, the treatment may be applies to only portions of the substrate surface. These treatments may be applied to materials that are suitable for use as the barrier zone or barrier material to prevent loss or accumulation of surfactant, for example, and wide range of nonwoven materials that can be used herein. Examples include all or any suitable portion of the chassis, the topsheet, the absorbent core, the core cover, the dusting layer/cover as described herein, the acquisition system, the barrier cuff, other absorbent article components, and/or any combination thereof. By way of example, suitable substrate materials for this treatment include, but are not limited to, nonwoven webs, cellulosic webs, thermoplastic films, modified/processed films (e.g., formed, apertured) and the like. Exemplary surface treatments using fluorocarbons are described in U.S. Pat. No. 5,876,753, issued to Timmons et al. on Mar. 2, 1999; U.S. Pat. No. 5,888,591 issued to Gleason et al. on Mar. 30, 1999; U.S. Pat. No. 6,045,877 issued to Gleason et al. on Apr. 4, 2000; PCT Patent Application 99/20504 by D'Agostino et al., published on Mar. 7, 1999; PCT Publication 00/14296 by D'Agostino et al., published on Mar. 16, 2000.

Other surface coating methods using silicones or fluoro chemicals are known in the art and may be used herein. The conventional coating or surface treatment methods typically fill the voids within the coated material, thereby lowering its air permeability. Coating methods to provide hydrophobicity to the substrate without the decrease in air permeability can be found, for example, in U.S. Pat. No. 5,322,729 and PCT Publication WO 96/03501.

In order to provide the desired “barrier-like” property, the stabilizing component that is a substantially permanent hydrophobic agent that serves as the barrier coating can comprise or be selected from one or more materials that substantially closes or block the openings (as opposed to the full length) of the pores of the portion of the barrier layer onto which it is deposited. More specifically, the barrier coating can comprise or be selected from thermoplastic materials (such as hotmelt adhesives), solutions, emulsions, dispersions, or any combination thereof In one aspect, for example, suitable barrier coating hydrophobic agents can comprise or be selected from microcrystalline waxes, stearyl behenates, sucrose fatty acids, polyisobutylenes, ethylene-vinyl acetate copolymer resins, polyethylene waxes, fatty alcohols, sucrose fatty acid esters, stearyl alcohol, sucrose hardened soy esters having an iodine value of less than 107 (typically less than 90), natural alcohol still bottoms, wax esters (e.g., sorbitan wax ester, fatty-fatty wax ester, sucrose wax esters), aldol condensation products with melting points greater than 60° C., natural petroleum waxes, lube base stocks, ozokerite wax, synthetic petroleum waxes, beeswax, stearic acids, spermaceti, carnauba wax, hydrogenated soybean oil, unhydrogenated soybean oil, corn oil, palm oil, coconut oil, castor oil, linseed oil, safflower oil, sunflower oil, rapeseed oil, silicones, xanthan gum, gum arabic, celluloses, chemical and enzyme-modified starches, petrolatums, mineral oils, vinyl copolymers, vinyl emulsifiers, sorbitol, propylene glycol, glycerine, solid esters, or any combination thereof. In a further aspect, the barrier coating can comprise or be selected from microcrystalline waxes, thermoplastic materials, stearyl behenates, stearyl alcohols, silicones, silicone waxes, or any combination thereof. The barrier coating can also comprise or be selected from microcrystalline waxes, hotmelt adhesives, or a combination thereof. Suitable hotmelt adhesives include, but are not limited to, H. B. Fuller's HL-1258.

In one aspect, the barrier coating can be a microcrystalline wax. One suitable microcrystalline wax for this use is Multiwax W-835, which is commercially available from The C.P. Hall Company. This material is a highly refined, high molecular weight microcrystalline petroleum wax that includes saturated branched and cyclic non-polar hydrocarbons.

As provided in this disclosure, the substantially permanent hydrophobic agent typically is not associated with the fibers of the nonwoven material in the same manner disclosed for the hydrophilizing agent, although the barrier material may constitute at least a partial coating or barrier material on the hydrophilic nonwoven material itself to reduce surfactant loss. In one aspect for example, when the disposable absorbent article contains a standing cuff, the hydrophobic agent can be applied to the standing cuff or disposed between the hydrophilic nonwoven material and the standing cuff, or even applied or disposed as a coating on the hydrophilic nonwoven material, in order to decrease loss of surfactant.

In a further aspect of stabilizing the nonwoven material to surfactant loss, the stabilizing component can be selected from at least one substantially permanent hydrophobic agent independent, separate, or apart from the surfactant-treated hydrophilic nonwoven material, i.e., this hydrophobic agent is not associated with the fibers of the surfactant-treated nonwoven material in the same manner that the hydrophilizing agent is associated with the fibers. Thus, a nonwoven fabric that includes nanofibers provides better barrier property compared to more conventional nonwoven fabrics. By using a nanofiber or nanofiber-containing nonwoven as a barrier, an absorbent article can maintain the desired moisture containment property, even if some surfactant migration occurs. In one aspect, the nanofiber or nanofiber-containing nonwoven can be used in a diaper as a barrier-on-core, outercover, and/or leg cuff. Additional disclosure relating to nanofiber utility and methods of making may be found in U.S. Pat. Appl. Pub. No. 2005/0070866, published Mar. 31, 2005.

In this aspect, the stabilizing component can be a substantially permanent hydrophobic agent, the agent comprising a nanofiber-containing nonwoven material, wherein: a) the nanofibers have an average diameter less than one micron; and b) nanofiber-containing nonwoven material has an average pore diameter less than about 15 microns and a coefficient of variation in pore size diameter less than about 20%.

The nanofibers typically have a diameter of less than about 1 micron, and typically comprise a significant number of the fibers, typically at least about 50%, in one layer of the nonwoven web in which they are included in the absorbent article. The nanofibers may be produced from a melt film fibrillation process, which generally includes the steps of providing a polymeric melt, utilizing a central fluid stream to form an elongated hollow polymeric film tube, and using this and/or other fluid streams to form multiple nanofibers from the hollow tube. Suitable nanofibers and their preparation are disclosed in U.S. Pat. Appl. Publ. No. 2005/0070866.

In yet another aspect, the nanofibers can be made from a melt film fibrillation process comprising the steps of: a) providing polymer in the form of a polymeric melt; b) utilizing a central fluid stream to form an elongated hollow polymeric film tube, c) using a fluid to form multiple nanofibers from the hollow polymeric film tube; and d) providing an orifice having a die collector distance which is optimized to obtain the coefficient of variation in pore size diameter of less than about 20%.

Useful nanofibers include those that can be produced from one or more thermoplastic polymers. Nonlimiting examples of thermoplastic polymers suitable for this application include polyolefins, polyesters, polyamides, polystyrenes, polyurethanes, biodegradable polymers including thermoplastic starch, PHA, PLA, starch compositions, and any combination thereof Homopolymers, copolymers, interpolymers (which includes terpolymers, etc.), and blends thereof are included within this description. In this aspect, useful polymers from which nanofibers can be produced include polyolefins such as polypropylene, polyethylene, nylons, polyethylene terephthalate, and the like.

Suitable thermoplastic polymers include any polymer suitable for melt spinning. The theological properties of the polymer can be such that the polymer can be melt extruded and is able to form a film. The melting temperature of the polymer can be generally from about 25° C. to about 400° C. The polymers of this aspect as they are present in the die may have a melt flow rate of less than about 400 decigrams per minute, as measured using ASTM method D-1238. In one aspect, the melt flow rate may be less than about 300 decigrams per minute, less than about 200 decigrams per minute, or less than about 100 decigrams per minute. A typical range of melt flow rates is from about 1 decigram per minute to about 100 decigrams per minute. Generally, lower melt flow rates within this range work well; therefore, polymers with melt flow rates less than about 50 decigrams per minute and less than about 40 decigrams per minute may be utilized.

The fibers may be single or multicomponent fibers such as bicomponent fibers. The fibers may have a sheath-core or side-by-side or other suitable geometric configuration. After the fibers are made, the fibers may be treated or coated before they are formed into a web, and/or after a web is made, the web itself may be treated. Thus, optional additives may be compounded into the polymer resin which may move out to the surface of the fiber after the fibers are formed, and may be used to alter or “tune” the properties of the fiber. Typically, any additives that migrate to the surface may need to be cured utilizing external energy, such as heat, or additives on surface may need to be chemically reacted with another component or curing may need to be catalyzed in the presence of another component, such that additional components may be added to the process while the fibers are being made or after the fibers are made using the resin with additives. Suitable treatments include treatments that can alter or tune the hydrophilic or hydrophobic properties of the fiber, an example of which is treatment with poly-di-methyl-siloxanes as a hydrophobic treatment. The specific treatment depends on the use of the web, type of polymer, and the like.

A useful method of making the nanofibers of the present disclosure is a melt fibrillation process, for example, a melt film fibrillation process. Generally, a melt film fibrillation process involves providing a polymeric melt, utilizing a central fluid stream to form an elongated hollow polymeric film tube, and then using a fluid to form multiple nanofibers from the hollow tube. Suitable methods are detailed, for example, in U.S. Pat. No. 4,536,361 to Torobin and U.S. Pat. Nos. 6,382,526 and 5,520,425 to Reneker. The melt film fibrillation methods can utilize various different processing conditions. Reneker's method more specifically includes the steps of feeding the polymer into an annular column and forming a film or tube at the exit of the annular column where a gas jet space is formed. A gas column then provides pressures on the inner circumference of the polymer tube. When the polymer tube exits the gas jet space, it is blown apart into many small fibers, including nanofibers, due to the expanding central gas.

Another example of a melt film fibrillation method that more specifically describes the steps of melting the polymer to form a polymeric melt, extruding the polymeric melt through an orifice which contains a central fluid stream such that the polymer extrudes as an elongated hollow tube, is found in U.S. Pat. Appl. Pub. No. 2005/0070866. The elongated hollow polymer tube can be circular, elliptical, irregular, or any other shape which has a hollow region. In some cases, the elongated hollow polymer tube may collapse immediately after forming. In the case of the collapsed tube, it may be useful to have thinned walls or weakened portions in the tube to aid in the fibrillation. Non-limiting examples of the fiberizing fluid are gases such as nitrogen or air. In one aspect, the fiberizing fluid can be at a temperature close to the temperature of the melted polymer. For example, the fiberizing fluid temperature may be a higher temperature than the melted polymer to help in the flow of the polymer and the formation of the hollow tube. Alternatively, the fiberizing fluid temperature can be below the melted polymer temperature to assist in the formation and solidification of the nanofibers. In one aspect, the fiberizing fluid temperature is less than the polymer melting point, for example, more than about 50° C. below the polymer melting point, or more than about 100° C. below the polymer melting point, or just at ambient temperature. The pressure of the fiberizing fluid is sufficient to fibrillate the nanofibers and can be slightly above the pressure of the melted polymer as it is extruded out of the orifice.

The nanofibers of this aspect of the disclosure are used to make nonwoven webs which can constitute the total nonwoven composite. This web may have one or several layers, which generally is described as the web or part of the web that is produced in a separate fiber lay down or forming step. These webs typically will comprise one or more layers having a significant number of nanofibers having diameters of less than one micron. In this aspect of the disclosure, a “significant number” is defined as at least about 25%. The significant number of fibers can be more than about 35%, more than about 50%, or more than about 75% of the total number of fibers in the layer. The web could have 100% of the fibers having a diameter of less than about one micron. The fiber diameters of the web can be measured using a scanning electron microscope at a magnification of greater than about 500 times and up to about 10,000 times as needed for visual analysis. To determine if a significant number of fibers have diameters less than one micron, typical measurement conditions include at least about 100 fibers or more fibers that can be measured, the measurements may occur at various regions throughout the layer, and sufficient sampling that is statistically significant occurs.

The fiber diameter of the remaining larger fibers, up to about 75%, may have fiber diameters in any range. Typically, the larger fiber diameters will be just above one micron to about 10 microns.

Further to this aspect, a significant number of fibers in a layer will have a fiber diameter of less than about 900 nanometers and typically from about 100 nanometers to about 900 nanometers. The fibers may have a diameter of less than 700 nanometers and from about 300 to about 900 nanometers. The diameters typically depend upon the desired use of the web. For process and product benefits, it may be desirable in some applications to have a significant number of fibers having a diameter of less than about one micron and a significant number of fibers having a diameter of greater than about one micron. The larger fibers may trap and immobilize the nanofibers, a feature that may help to reduce the amount of clumping or roping of the nanofibers and prevent the nanofibers from being carried off by stray air currents.

In one aspect, the nanofibers can have an average diameter less than about one micron, wherein the hydrophilic nonwoven material and associated hydrophilizing agent has an average pore diameter of less than about 15 microns and a coefficient of variation in pore size diameter less than about 20%, and wherein the nanofibers are made from a melt film fibrillation process comprising the steps of: a) providing polymer in the form of a polymeric melt; b) utilizing a central fluid stream to form an elongated hollow polymeric film tube, c) using a fluid to form multiple nanofibers from the hollow polymeric film tube; and d) providing an orifice having a die collector distance which is optimized to obtain the coefficient of variation in pore size diameter of less than about 20%.

The layers of nanofibers in a web of the present disclosure may contain more than one polymer. Moreover, different polymers or polymer blends may be used for different orifices to produce layers in a web having different fiber diameters and different polymer compositions. It may be desirable to produce a single layer nonwoven with varying fiber diameters. Alternatively, it may be desirable to produce a nonwoven web with multiple layers with each layer having different fiber diameters. The melt film fibrillation process can be modified to produce both small and large diameter fibers to make various webs. These and other aspects of producing the nonwoven web containing nanofibers are found in U.S. Pat. Appl. Pub. No. 2005/0070866.

In a diaper or other absorbent article, the web may be used as a barrier layer as disclosed herein. These nanofiber-containing webs may also be used as a high barrier cuff with a high hydrostatic head to enable low leakage incident rates of thin, narrow crotch diapers desired for comfort and fit, and as a barrier such that the absorbent article can maintain the desired moisture containment property even if some surfactant migration occurs. For example, in a diaper or other disposable absorbent product, the nanofiber-containing nonwoven web may be utilized as a barrier layer. This barrier layer may be disposed between the absorbent core that is primarily responsible for fluid handling properties such as acquiring, transporting, distributing, and storing body fluids and an outer layer of the disposable absorbent product. The barrier layer may also or alternatively be disposed between the absorbent core and the topsheet, it also may be absorbent, and typically it has a balance between convective air flow and an absorptive barrier property. The convective air flow property is effective to reduce the relative humidity within the space between the absorbent article and the wearer's skin. The combination of liquid absorption and liquid barrier property provides protection against the wet through problem and is especially beneficial when the absorbent article is under impact and/or sustained pressure. Further description and benefits of the barrier layers may be found in WO 01/97731.

Under surfactant migration conditions, when sufficient surfactant leaves the designed hydrophilic material and migrates such that the designed hydrophilic material approaches or becomes hydrophobic, activation of a wetness indicator may not result, even though sufficient wetness has been retained by the absorbent article to activation the wetness indicator according to its intended design. A wetness indicator can be disposed between the absorbent core and the backsheet, for example in a nonwoven dusting layer/cover, yet surfactant migration from the nonwoven dusting layer/cover into the cuff, core, etc. can result in the dusting layer/cover showing difficulty in allowing liquid through to a wetness indicator. However, wetness indicators can be combined with the stabilizing components of this disclosure to afford a wetness indicator design that functions as designed, by encouraging liquid to migrate toward the wetness indicator rather than following the migrating surfactant to a standing cuff or other portions of the absorbent article.

Accordingly, the disposable absorbent articles of the present disclosure may further comprise a wetness indicator disposed between the absorbent core and the backsheet and in liquid communication with the absorbent core. In one aspect, the wetness indicator can comprise a central graphic and a background graphic wherein the background graphic comprises at least one responsive color composition that, upon wetting, exhibits a visible change. For example, the visible change can be a color change, a graphic change, or combinations thereof In certain aspects, the central graphic is hidden by the background graphic until such time as the graphic is wetted. Examples of suitable wetness indicators are provided in U.S. Pat. No. 7,332,642 and EP 1,242,027.

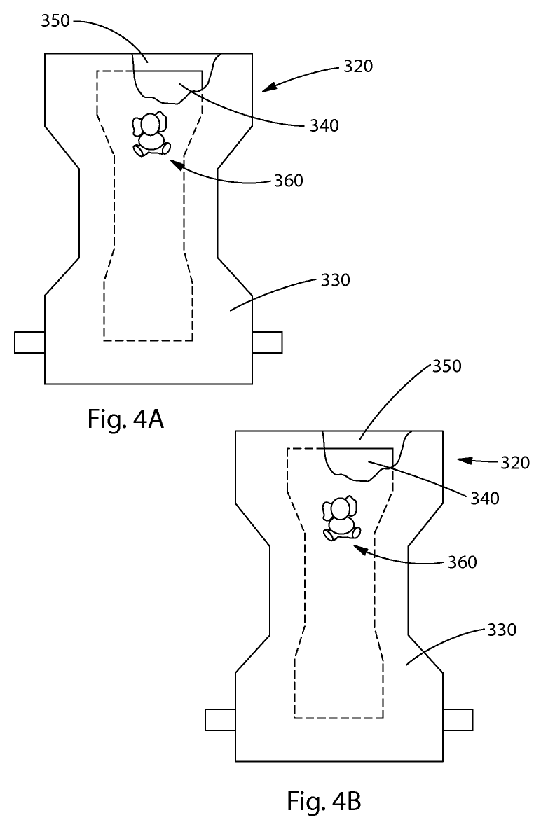

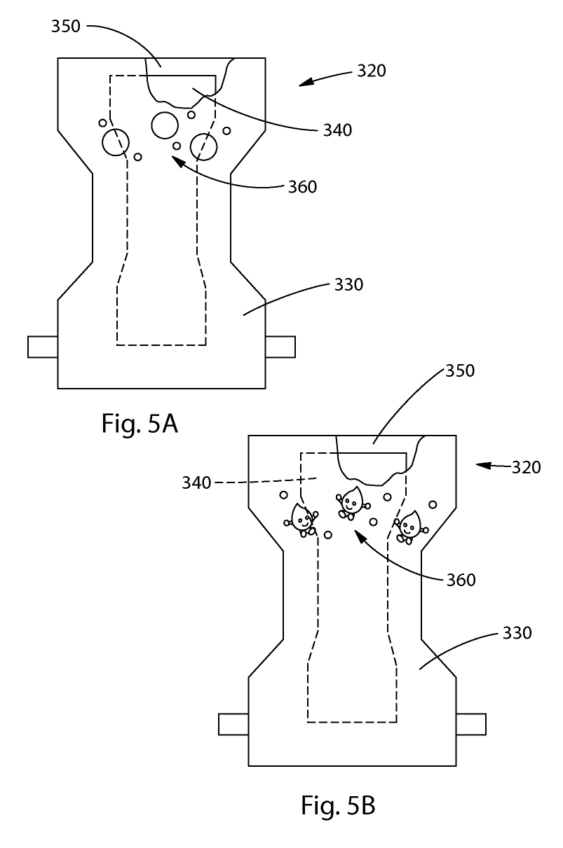

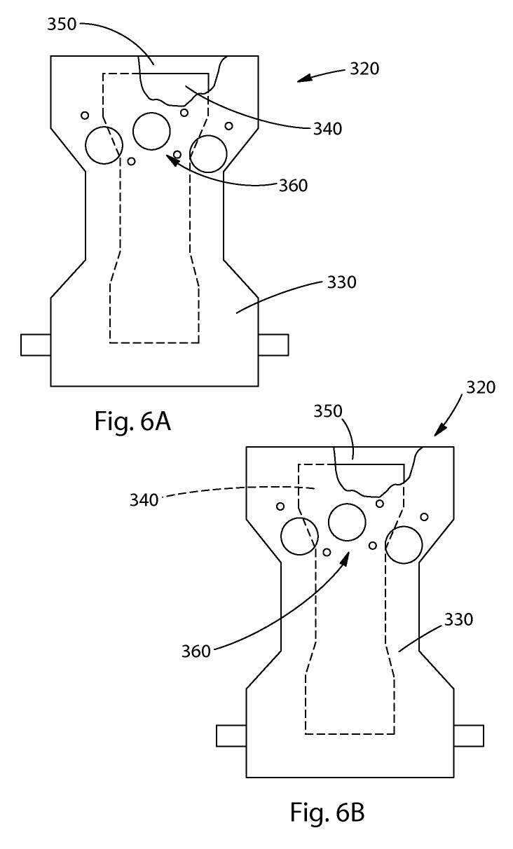

For instance, FIGS. 4A and 4B illustrate an absorbent article 320 of the present disclosure comprising a topsheet 330, an absorbent core 340, and backsheet 350 wherein the wetness indicator 360 includes a background graphic of a colored animal that once wetted (shown in 4B) reveals a central graphic of a different colored animal. FIGS. 5A and 5B depict an absorbent article of the present disclosure wherein the wetness indicator includes a central graphic of colored raindrops that are revealed upon wetting of a colored larger animated bubbles background graphic. In other aspects, the background graphic may be a different color than the central graphic that is revealed upon wetting which can indicate the difference in the wetness indicator pre- and post-wetting. Similarly, FIGS. 6A and 6B illustrate that the wetness indicator's background graphic may be wetted to reveal a central graphic that is of an entirely or of a partially different color from the background graphic. For instance, each of the larger and smaller bubbles in FIG. 6A prior to wetting may be blue in color but after wetting (as shown in FIG. 6B) only the smaller sized bubbles may change to pink in color while the raindrops within the larger bubbles may appear pink in color while the surrounding larger bubbles remains blue in color after wetting. Alternatively, prior to wetting each of the bubbles may be blue in color and upon wetting the raindrops that are revealed may each be a different color from the original blue bubbles and may still be different colors from one another.

According to the present disclosure, a responsive color composition may comprise from about 1% to about 10%, by weight of the composition, of a pigment. Further, the composition may comprise from about 2% to about 8% or from about 3% to about 7% of the pigment.

Solid pigments particles that may be used in the wetness indicator include, but are not limited to, for example, pigment Yellow (C.I. 14), pigment Red (C.I. 48:3), pigment Blue (C.I. 15:4), pigment Black (C.I. 7), or any combinations thereof.

The responsive color composition also can comprise from about 1% to about 10%, by weight of the composition, of a fluid dyestuff. In other aspects, the composition comprises from about 2% to about 8% or from about 3% to about 7% of the fluid dyestuff.

Suitable fluid dyestuffs include water soluble ink colorants like direct dyes, acid dyes, base dyes, and various solvent soluble dyes and the like. Examples include, but are not limited to, FD&C Blue 1 (C.I. 42090:2), D&C Red 6(C.I. 15850), D&C Red 7(C.I. 15850:1), D&C Red 9(C.I. 15585:1), D&C Red 21(C.I. 45380:2), D&C Red 22(C.I. 45380:3), D&C Red 27(C.I. 45410:1), D&C Red 28(C.I. 45410:2), D&C Red 30(C.I. 73360), D&C Red 33(C.I. 17200), D&C Red 34(C.I. 15880:1), and FD&C Yellow 5(C.I. 19140:1), FD&C Yellow 6(C.I. 15985:1), FD&C Yellow 10(C.I. 47005:1), D&C Orange 5(C.I. 45370:2), and any combination thereof.

Moreover, the responsive color composition can comprise from about 10% to about 99% of a solvent. In one aspect, the composition comprises from about 30% to about 85% and in another aspect from about 40% to about 75% of a solvent.

The solvents that can be used in the wetness indicators disclosed herein may be selected from water, non-aqueous solvents, or any combinations thereof. For example, non-aqueous solvents that are useful include alcohols, acetates, and combinations thereof. Examples of alcohol solvents include, but are not limited to, iso-propyl alcohol, n-propyl alcohol, ethanol, methanol, and combinations thereof. Likewise, suitable acetate solvents include, but are not limited to, isopropyl acetate, n-propyl acetate, and combinations thereof.

An exemplary ink that can be used in the wetness indicator to dispose both the central and background graphics on a substrate is commercially available from Osaka Printing Industries as Omutsu-you-G ICC pants-you Hkonku v/M (translated from Japanese to English).

In an alternative aspect, the responsive color composition comprises from about 1% to about 10%, by weight of the composition, of a fluid dyestuff and from about 10% to about 99%, by weight of the composition, of a solvent. In the absence of a pigment in the responsive color composition, such a composition may be disposed adjacent to a varnish coating, which is detailed in U.S. Pat. No. 7,332,642. In this aspect, the responsive color composition comprises from about 2% to about 8% of a fluid dyestuff, typically from about 2.5% to about 7% or from about 3% to about 6%. Additionally, such a composition can comprise from about 30% to about 80% of a solvent or from about 40% to about 75% of a solvent.

The central graphic of the wetness indicator disclosed herein may additionally comprise additional responsive color compositions that include the same or similar constituents as the first responsive color composition.

In a further aspect, the articles of the present disclosure may further comprise a permanent color composition, particular in the wetness indicator. This permanent color composition comprises from about 1% to about 10%, by weight of the composition, of a pigment. Suitable pigments include those that are provided relative to the responsive color composition. In this aspect, the permanent color composition may additionally comprise a non-aqueous solvent in an amount of from about 10% to about 99%, from about 30% to about 80%, or from about 40% to about 75%. Again, those solvents that are suitable for this aspect are similar to those disclosed herein for the responsive color composition.

Another example of suitable wetness indicators is provided in EP 1,242,027. In this aspect, the indicator can be printed on an impermeable polymer backsheet that changes its color when urine or moisture reaches the indicator.

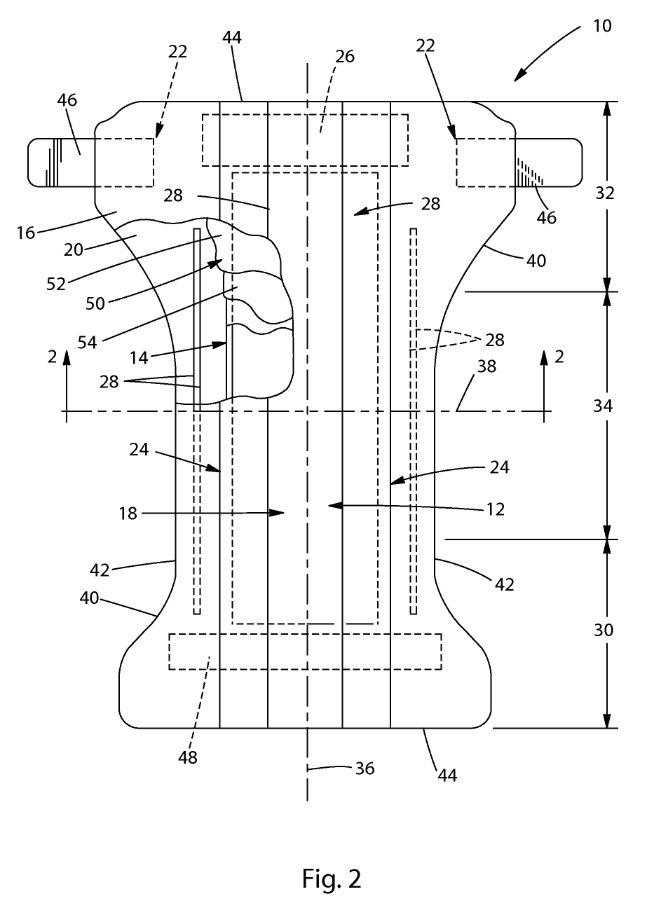

FIG. 2 is a plan view of a diaper 10 according to a certain aspect of the present disclosure in which the stabilizing components and wetness indicators of this disclosure may be employed. Diaper 10 is shown in its flat out, uncontracted state (i.e., without elastic induced contraction) and portions of the diaper 10 are cut away to more clearly show the underlying structure of the diaper 10. A portion of the diaper 10 that contacts a wearer is facing the viewer in FIG. 2. The diaper 10 generally may comprise a chassis 12 and an absorbent core 14 disposed in the chassis.

The chassis 12 of the diaper 10 in FIG. 2 may comprise the main body of the diaper 10. The chassis 12 may comprise an outer covering 16 including a topsheet 18, which may be liquid pervious, and/or a backsheet 20, which may be liquid impervious. The absorbent core 14 may be encased between the topsheet 18 and the backsheet 20. The chassis 12 may also include side panels 22, elasticized leg cuffs 24, and/or an elastic waist feature 26.

The leg cuffs 24 and the elastic waist feature 26 may each typically comprise elastic members 28. One end portion of the diaper 10 may be configured as a first waist region 30 of the diaper 10. An opposite end portion of the diaper 10 may be configured as a second waist region 32 of the diaper 10. An intermediate portion of the diaper 10 may be configured as a crotch region 34, which extends longitudinally between the first and second waist regions 30 and 32. The waist regions 30 and 32 may include elastic elements such that they gather about the waist of the wearer to provide improved fit and containment (elastic waist feature 26). The crotch region 34 is that portion of the diaper 10 which, when the diaper 10 is worn, is generally positioned between the wearer's legs.

The diaper 10 is depicted in FIG. 2 with its longitudinal axis 36 and its transverse axis 38. The periphery 40 of the diaper 10 is defined by the outer edges of the diaper 10 in which the longitudinal edges 42 run generally parallel to the longitudinal axis 36 of the diaper 10 and the elastic end edges 44 run between the longitudinal edges 42 generally parallel to the transverse axis 38 of the diaper 10. The chassis 12 may also comprise a fastening system, which may include at least one fastening member 46 and at least one stored landing zone 48.

The diaper 10 may also include such other features as are known in the art including front and rear ear panels, waist cap features, elastics and the like to provide better fit, containment and aesthetic characteristics. Such additional features are well known in the art and are described in, e.g., U.S. Pat. No. 3,860,003 and U.S. Pat. No. 5,151,092.

In order to keep the diaper 10 in place about the wearer, at least a portion of the first waist region 30 may be attached by the fastening member 46 to at least a portion of the second waist region 32 to form leg opening(s) and an article waist. When fastened, the fastening system carries a tensile load around the article waist. The fastening system may allow an article user to hold one element of the fastening system, such as the fastening member 46, and connect the first waist region 30 to the second waist region 32 in at least two places. This may be achieved through manipulation of bond strengths between the fastening device elements.

According to certain aspects, the diaper 10 may be provided with a re-closable fastening system or may alternatively be provided in the form of a pant-type diaper. When the absorbent article is a diaper, it may comprise a re-closable fastening system joined to the chassis for securing the diaper to a wearer. When the absorbent article is a pant-type diaper, the article may comprise at least two side panels joined to the chassis and to each other to form a pant. The fastening system and any component thereof may include any material suitable for such a use, including but not limited to plastics, films, foams, nonwoven, woven, paper, laminates, fiber reinforced plastics and the like, or combinations thereof. In some aspects, the materials making up the fastening device may be flexible. The flexibility may allow the fastening system to conform to the shape of the body and thus, reduce the likelihood that the fastening system will irritate or injure the wearer's skin.

For unitary absorbent articles, the chassis 12 and absorbent core 14 may form the main structure of the diaper 10 with other features added to form the composite diaper structure. While the topsheet 18, the backsheet 20, and the absorbent core 14 may be assembled in a variety of well-known configurations, for example, diaper configurations are described generally in U.S. Pat. No. 5,554,145 entitled “Absorbent Article With Multiple Zone Structural Elastic-Like Film Web Extensible Waist Feature” issued to Roe et al. on Sep. 10, 1996; U.S. Pat. No. 5,569,234 entitled “Disposable Pull-On Pant” issued to Buell et al. on Oct. 29, 1996; and U.S. Pat. No. 6,004,306 entitled “Absorbent Article With Multi-Directional Extensible Side Panels” issued to Robles et al. on Dec. 21, 1999.

The topsheet 18 in FIG. 2 may be fully or partially elasticized or may be foreshortened to provide a void space between the topsheet 18 and the absorbent core 14. Exemplary structures including elasticized or foreshortened topsheets are described in more detail in U.S. Pat. No. 5,037,416 entitled “Disposable Absorbent Article Having Elastically Extensible Topsheet” issued to Allen et al. on Aug. 6, 1991; and U.S. Pat. No. 5,269,775 entitled “Trisection Topsheets for Disposable Absorbent Articles and Disposable Absorbent Articles Having Such Trisection Topsheets” issued to Freeland et al. on Dec. 14, 1993.

The backsheet 20 may be joined with the topsheet 18. The backsheet 20 may prevent the exudates absorbed by the absorbent core 14 and contained within the diaper 10 from soiling other external articles that may contact the diaper 10, such as bed sheets and undergarments. In certain aspects, the backsheet 26 may be substantially impervious to liquids (e.g., urine) and comprise a laminate of a nonwoven and a thin plastic film such as a thermoplastic film having a thickness of about 0.012 mm (0.5 mil) to about 0.051 mm (2.0 mils). Suitable backsheet films include those manufactured by Tredegar Industries Inc. of Terre Haute, Ind. and sold under the trade names X15306, X10962, and X10964. Other suitable backsheet materials may include breathable materials that permit vapors to escape from the diaper 10 while still preventing exudates from passing through the backsheet 10. Exemplary breathable materials may include materials such as woven webs, nonwoven webs, composite materials such as film-coated nonwoven webs, and microporous films such as manufactured by Mitsui Toatsu Co., of Japan under the designation ESPOIR NO and by EXXON Chemical Co., of Bay City, Tex., under the designation EXXAIRE. Suitable breathable composite materials comprising polymer blends are available from Clopay Corporation, Cincinnati, Ohio under the name HYTREL blend P18-3097. Such breathable composite materials are described in greater detail in PCT Application No. WO 95/16746, published on Jun. 22, 1995 in the name of E. I. DuPont. Other breathable backsheets including nonwoven webs and apertured formed films are described in U.S. Pat. No. 5,571,096 issued to Dobrin et al. on Nov. 5, 1996.

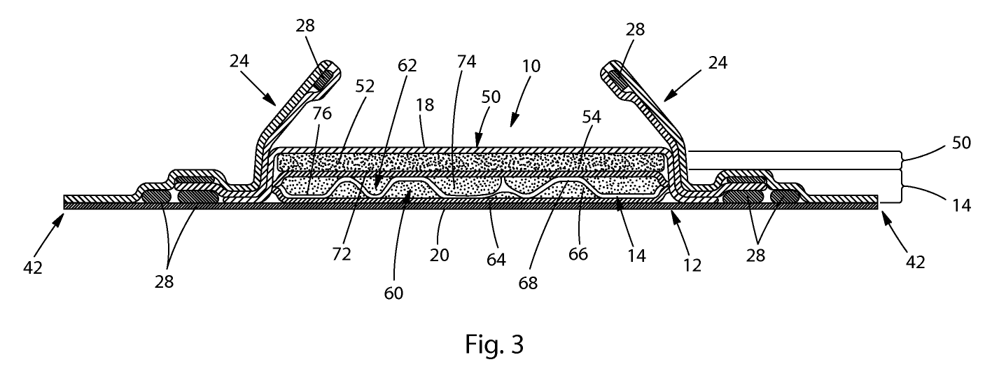

FIG. 3 shows a cross section of FIG. 2 taken along the sectional line 2-2 of FIG. 2. Starting from the wearer facing side, the diaper 10 may comprise the topsheet 18, the components of the absorbent core 14, and the backsheet 20. According to a certain aspect, diaper 10 may also comprise an acquisition system 50 disposed between the liquid permeable topsheet 18 and a wearer facing side of the absorbent core 14. The acquisition system 50 may be in direct contact with the absorbent core. The acquisition system 50 may comprise a single layer or multiple layers, such as an upper acquisition layer 52 facing towards the wearer's skin and a lower acquisition 54 layer facing the garment of the wearer. According to another aspect, the acquisition system 50 may function to receive a surge of liquid, such as a gush of urine. In other words, the acquisition system 50 may serve as a temporary reservoir for liquid until the absorbent core 14 can absorb the liquid.

In certain aspects, the acquisition system 50 may comprise chemically cross-linked cellulosic fibers. Exemplary chemically cross-linked cellulosic fibers are disclosed in U.S. Pat. No. 5,137,537. One or both of the upper and lower acquisition layers 52 and 54 may comprise a nonwoven, which may be hydrophilic. Further, according to this disclosure, one or both of the upper and lower acquisition layers 52 and 54 may comprise the chemically cross-linked cellulosic fibers, which may or may not form part of a nonwoven material.

Suitable nonwoven materials for the upper and lower acquisition layers 52 and 54 include, but are not limited to SMS material, comprising a spunbonded, a melt-blown and a further spunbonded layer. In certain aspects, permanently hydrophilic nonwovens, and in particular, nonwovens with durably hydrophilic coatings are used. Another suitable material comprises a SMMS-structure. In another aspect, the nonwovens can be porous.

According to a further aspect, suitable nonwoven materials may include but are not limited to synthetic fibers, such as PE, PET, and PP. As polymers used for nonwoven production may be inherently hydrophobic, they may be coated with hydrophilic coatings. Moreover, such nonwovens may be treated with surfactants to enhance their hydrophilic character, and the stabilizing components and methods disclosed herein address the stability of such surfactant-treated materials toward loss, leaching, or elution of surfactants during wetting.

One way to produce nonwovens with durably hydrophilic coatings, is by applying a hydrophilic monomer and a radical polymerization initiator onto the nonwoven, and conducting a polymerization activated via UV light resulting in monomer chemically bound to the surface of the nonwoven as described in U.S. Patent Publication No. 2005/0159720. Another way to produce nonwovens with durably hydrophilic coatings is to coat the nonwoven with hydrophilic nanoparticles as described in U.S. Pat. No. 7,112,621 to Rohrbaugh et al. and in PCT Application Publication WO 02/064877.

Further useful nonwovens are described in U.S. Pat. No. 6,645,569 to Cramer et al., U.S. Pat. No. 6,863,933 to Cramer et al., U.S. Pat. No. 7,112,621 to Rohrbaugh et al., and U.S. Pat. Appl. Pub. Nos. 20030148684 to Cramer et al. and 20050008839 to Cramer et al.

In some cases, the nonwoven surface can be pre-treated with high energy treatment (corona, plasma) prior to application of nanoparticle coatings. High energy pre-treatment typically temporarily increases the surface energy of a low surface energy surface (such as PP) and thus enables better wetting of a nonwoven by the nanoparticle dispersion in water.

Notably, permanently hydrophilic nonwovens are also useful in other parts of an absorbent article. For example, topsheets and absorbent core layers comprising permanently hydrophilic nonwovens as described above have been found to work well.

The absorbent core 14 generally is disposed between the topsheet 18 and the backsheet 20 and may comprise two layers, a first absorbent layer 60 and a second absorbent layer 62. The first absorbent layer 60 of the absorbent core 14 comprises a first substrate 64, an absorbent particular polymer material 66 on the first substrate 64, and a thermoplastic composition 68 on the absorbent polymer material 66 and at least portions of the first substrate 64 as an adhesive for covering and immobilizing the absorbent polymer material 66 on the first substrate 64. According to another aspect of this invention, the first absorbent layer 60 of the absorbent core 14 may also include a cover layer on the thermoplastic composition 68. The thermoplastic composition may be a thermoplastic adhesive material which as used herein is understood to comprise a polymer composition from which fibers are formed and applied to the superabsorbent material with the intent to immobilize the superabsorbent material in both the dry and wet state. The thermoplastic adhesive material of the present invention forms a fibrous network over the superabsorbent material.

Likewise, as best illustrated in FIG. 3, the second absorbent layer 62 of the absorbent core 14 may also include a second substrate 72, an absorbent polymer material 74 on the second substrate 72, and a thermoplastic composition 76 on the absorbent polymer material 74 and at least a portion of the second substrate 72 for immobilizing the absorbent polymer material 74 on the second substrate 72. Although not illustrated, the second absorbent layer 62 may also include a cover layer.

The substrate 64 of the first absorbent layer 60 may be referred to as a dusting layer/cover and has a first surface which faces the backsheet 20 of the diaper 10 and a second surface which faces the absorbent polymer material 66. Likewise, the substrate 72 of the second absorbent layer 62 may be referred to as a core cover and has a first surface facing the topsheet 18 of the diaper 10 and a second surface facing the absorbent polymer material 74. The first and second substrates 64 and 72 may be adhered to one another with adhesive about the periphery to form an envelope about the absorbent polymer materials 66 and 74 to hold the absorbent polymer material 66 and 74 within the absorbent core 14.

According to other aspects, the substrates 64 and 72 of the first and second absorbent layers 60 and 62 may be a nonwoven material, such as those nonwoven materials described above.

Exemplary absorbent structures for use as the absorbent assemblies are described in U.S. Pat. No. 4,610,678 (Weisman et al.); U.S. Pat. No. 4,834,735 (Alemany et al.); U.S. Pat. No. 4,888,231 (Angstadt); U.S. Pat. No. 5,260,345 (DesMarais et al.); U.S. Pat. No. 5,387,207 (Dyer et al.); U.S. Pat. No. 5,397,316 (LaVon et al.); and U.S. Pat. No. 5,625,222 (DesMarais et al.).

In the nonwoven materials provided in this basic design, particularly the nonwovens in the core, surfactant migration during wetting can cause contamination and wetting in other parts of the absorbent article, e.g. in the standing cuff. Thus, when surfactant migrates or diffuses from the core throughout the absorbent article to materials that selected for their hydrophobic barrier properties, such as the standing cuff, these hydrophobic materials may become hydrophilic thereby reducing the performance of the article by loss of their barrier properties. The methods and structures provided herein can address this issue, and can be applied to any suitable materials disclosed within the basic diaper design disclosed above. Thus, the methods and structures provided herein can prevent contamination through migration. These methods and structures also can be applicable to any of the other absorbent articles disclosed herein.

All patents and patent applications (including any patents which issue thereon) assigned to the Procter & Gamble Company referred to herein are hereby incorporated by reference to the extent that it is consistent herewith.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A disposable absorbent article, comprising:
a) a chassis comprising a liquid pervious topsheet and a liquid impervious backsheet that is at least partially joined to the topsheet; and
b) an absorbent core disposed at least partially between the topsheet and the backsheet and comprising an absorbent polymer material;
wherein the disposable absorbent article or any component thereof comprises a hydrophilic nonwoven material comprising a plurality of surfactant-treated synthetic fibers;
the disposable absorbent article further comprising:
c) a stabilizing component which reduces the loss of surfactant from the hydrophilic nonwoven material during wetting, the stabilizing component selected from:
i) at least one substantially permanent hydrophilizing agent associated with the hydrophilic nonwoven material;
ii) at least one substantially permanent hydrophobic agent independent of the hydrophilic nonwoven material, or
iii) a combination of i) and ii).

2. A disposable absorbent article according to claim 1, further comprising:
a) a core cover contiguous with the absorbent core and disposed between the topsheet and the absorbent core;
b) a dusting cover contiguous with the absorbent core and disposed between the backsheet and the absorbent core;
c) an acquisition system disposed between the topsheet and the absorbent core, or
d) any combination thereof.

3. A disposable absorbent article according to claim 1, wherein:
the stabilizing component is a substantially permanent hydrophilizing agent; and
the one or more of the synthetic fibers comprises a polymer and the hydrophilizing agent, wherein the polymer and the hydrophilizing agent comprise complementary segments that are associated with one another.

4. A disposable absorbent article according to claim 1, wherein the stabilizing component is a substantially permanent hydrophobic agent selected from a fluorocarbon compound, a silicone, a silicone wax, a thermoplastic material, a hotmelt adhesive, a microcrystalline wax, a stearyl behenate, a sucrose fatty acid, a polyisobutylene, an ethylene-vinyl acetate copolymer resin, a polyethylene wax, a fatty alcohol, a sucrose fatty acid ester, a stearyl alcohol, a sucrose hardened soy ester having an iodine value of less than 107, natural alcohol still bottoms, a wax ester, sorbitan wax ester, fatty-fatty wax ester, a sucrose wax ester, an aldol condensation product with a melting point greater than about 60° C., a natural petroleum wax, a lube base stock, ozokerite wax, a synthetic petroleum wax, beeswax, a stearic acid, spermaceti, carnauba wax, hydrogenated soybean oil, unhydrogenated soybean oil, corn oil, palm oil, coconut oil, castor oil, linseed oil, safflower oil, sunflower oil, rapeseed oil, xanthan gum, gum arabic, a cellulose, a chemically-modified starch, an enzyme-modified starch, a petrolatum, a mineral oil, a vinyl copolymer, a vinyl emulsifier, sorbitol, propylene glycol, glycerine, a solid ester, or any combinations thereof

5. A disposable absorbent article according to claim 1, wherein the disposable absorbent article further comprises a standing cuff, and the hydrophobic agent is applied to the standing cuff or disposed between the hydrophilic nonwoven material and the standing cuff.

6. A disposable absorbent article according to claim 1, wherein the stabilizing component is a substantially permanent hydrophobic agent, the agent comprising a nanofiber-containing nonwoven material, wherein:
a) the nanofibers have an average diameter less than one micron; and
b) nanofiber-containing nonwoven material has an average pore diameter less than about 15 microns and a coefficient of variation in pore size diameter less than about 20%.

7. A disposable absorbent article according to claim 6, wherein the nanofibers are made from a melt film fibrillation process comprising the steps of:
a) providing polymer in the form of a polymeric melt;
b) utilizing a central fluid stream to form an elongated hollow polymeric film tube,
c) using a fluid to form multiple nanofibers from the hollow polymeric film tube; and
d) providing an orifice having a die collector distance which is optimized to obtain the coefficient of variation in pore size diameter of less than about 20%.

8. The disposable absorbent article of claim 1, wherein the absorbent article is a diaper comprising a re-closable fastening system joined to the chassis for securing the diaper to a wearer.

9. The disposable absorbent article of claim 1, wherein the absorbent article is a pant-type diaper comprising at least two side panels joined to the chassis and to each other to form a pant.

10. The disposable absorbent article of claim 1, wherein the hydrophilic nonwoven material and the stabilizing component prevent contamination through migration.

11. A method of a stabilizing surfactant-treated synthetic fibers to surfactant loss during wetting, the method comprising:
a) providing a disposable absorbent article comprising a hydrophilic nonwoven material, the nonwoven material comprising surfactant-treated synthetic fibers;
b) combining or otherwise associating at least one substantially permanent hydrophilizing agent with the hydrophilic nonwoven material; and/or
c) depositing at least one substantially permanent hydrophobic agent on a portion of the disposable absorbent article independent of the hydrophilic nonwoven material.

12. A method of a stabilizing surfactant-treated synthetic fibers to surfactant loss during wetting according to claim 11, the method further comprising:
d) disposing a wetness indicator between the absorbent core and the backsheet and in liquid communication with the absorbent core, the wetness indicator comprises at least one responsive color composition such that, upon wetting, a visible change occurs.

13. A disposable absorbent article, comprising:
a) a chassis comprising a liquid pervious topsheet and a liquid impervious backsheet that is at least partially joined to the topsheet;
b) an absorbent core disposed at least partially between the topsheet and the backsheet and comprising an absorbent polymer material;
wherein the disposable absorbent article or any component thereof comprises a hydrophilic nonwoven material comprising a plurality of surfactant-treated synthetic fibers;
the disposable absorbent article further comprising:
c) a stabilizing component which reduces the loss of surfactant from the hydrophilic nonwoven material during wetting, the stabilizing component selected from:
i) at least one substantially permanent hydrophilizing agent associated with the hydrophilic nonwoven material;
ii) at least one substantially permanent hydrophobic agent independent of the hydrophilic nonwoven material, or
iii) a combination of i) and ii); and
d) a wetness indicator disposed between the absorbent core and the backsheet and in liquid communication with the absorbent core, the wetness indicator comprises at least one responsive color composition such that, upon wetting, a visible change occurs.

14. A disposable absorbent article according to claim 13, wherein the wetness indicator further comprises a hidden central graphic and a background graphic, the hidden central graphic comprises a permanent color composition and the background graphic comprises the at least one responsive color composition such that, upon wetting, the background graphic exhibits a visible change resulting in the hidden central graphic being revealed.

15. A disposable absorbent article according to claim 13, further comprising:
a) a core cover contiguous with the absorbent core and disposed between the topsheet and the absorbent core;
b) a dusting cover contiguous with the absorbent core and disposed between the backsheet and the absorbent core;
c) an acquisition system disposed between the topsheet and the absorbent core, or
d) any combination thereof.

16. A disposable absorbent article according to claim 13, wherein:
the stabilizing component is a substantially permanent hydrophilizing agent; and
the one or more of the synthetic fibers comprises a polymer and the hydrophilizing agent, wherein the polymer and the hydrophilizing agent comprise complementary segments that are associated with one another.

17. A disposable absorbent article according to claim 13, wherein the disposable absorbent article further comprises a standing cuff, and the hydrophobic agent is applied to the standing cuff or disposed between the hydrophilic nonwoven material and the standing cuff.

18. A disposable absorbent article according to claim 13, wherein the stabilizing component a substantially permanent hydrophobic agent, the agent comprising a nanofiber-containing nonwoven material, wherein:
a) the nanofibers have an average diameter less than one micron; and
b) nanofiber-containing nonwoven material has an average pore diameter less than about 15 microns and a coefficient of variation in pore size diameter less than about 20%.

19. A disposable absorbent article according to claim 13, wherein the nanofibers are made from a melt film fibrillation process comprising the steps of:
a) providing polymer in the form of a polymeric melt;
b) utilizing a central fluid stream to form an elongated hollow polymeric film tube,
c) using a fluid to form multiple nanofibers from the hollow polymeric film tube; and
d) providing an orifice having a die collector distance which is optimized to obtain the coefficient of variation in pore size diameter of less than about 20%.
